(19) United States
(12) Patent Application Publication
(10) Pub. No.: US 2021/0038863 A1
(43) Pub. Date: Feb. 11, 2021

(54) MEDICAL DEVICE HUB

(71) Applicant: Medtronic Vascular, Inc., Santa Rosa, CA (US)

(72) Inventors: Shane Cooney, Galway (IE); Stephen Nash, Clonellan (IE); Colm Connolly, Galway (IE); David Gannon, Galway (IE); Cian Michael Ryan, Galway (IE); Francis Denis McEvoy, Laois (IE)

(21) Appl. No.: 16/535,320

(22) Filed: Aug. 8, 2019

(51) Int. Cl. A61M 25/00; A61M 25/02

(52) U.S. Cl. CPC A61M 25/0097; A61M 2025/0253; A61M 25/02

(57) ABSTRACT

In some examples, a medical device includes an elongated body having an elongated body proximal end and a hub attached to the elongated body proximal end. The hub is configured to provide a removable attachment to an object, such as a surgical drape within a sterile field boundary. In some examples, the attachment mechanism comprises a resiliently biased first elongate arm and second elongate arm configured to establish a clamping action on the object and/or configured to establish a snap fit with each other while the objection is positioned between the first and second elongate arms. In some examples, the attachment mechanism may comprise a surface of the hub configured to provide a traction force on the object to hold the hub in place relative to the object.

### TECHNICAL FIELD

The disclosure relates to a hub for a medical device.

### BACKGROUND

Multiple medical devices may be simultaneously positioned in a lumen of a catheter during certain medical procedures, such as percutaneous coronary intervention procedures. These procedures can result in multiple medical devices proximally extending from a proximal end of a catheter. For example, in certain percutaneous coronary intervention procedures, two or more of a guidewire, a guide extension catheter, a balloon catheter, or a stent delivery device may be introduced into vasculature of a patient through the same lumen of an outer catheter.

### SUMMARY

In some examples, this disclosure describes a medical device that includes an elongated body having an elongated body proximal end, and a hub attached to the elongated body proximal end. The hub is configured to provide a removable attachment of the elongated proximal end to an object, such as a surgical drape within a sterile field boundary. For example, the hub may comprise an attachment mechanism that is configured to attach the hub to the object by holding a part of the object via a mechanical grasp or via a traction force.

A system may comprise the medical device and a catheter such as an outer catheter defining a lumen configured to receive the medical device. In addition, in some examples, a system may comprise a plurality of medical devices, with each medical device comprising an elongated member and a hub attached to the respective elongated member proximal end. Each hub may have an identifying visual characteristic such, but not limited to, one or more of a shape, a color, a size, a label, an alphanumeric indication, a graphical indication, or other characteristic.

This disclosure also describes example methods of using the system.

Clause 1: In some examples, a medical device comprises an elongated body comprising a proximal portion, and a hub attached to a proximal end of the proximal portion, the hub comprising an attachment mechanism configured to provide a removable attachment of the hub to an object.

Clause 2: In some examples of the medical device of clause 1, the attachment mechanism is configured to provide a nonperforating attachment to the object.

Clause 3: In some examples of the medical device of clause 1 or 2, the elongated body comprises a guide extension catheter and the proximal portion comprises a push member.

Clause 4: In some examples of the medical device of any of clauses 1-3, the hub surrounds the proximal end of the proximal portion.

Clause 5: In some examples of the medical device of any of clauses 1-4, the attachment mechanism comprises a first elongate arm and a second elongate arm movable relative to the first elongate arm.

Clause 6: In some examples of the medical device of clause 5, the first elongate arm is resiliently biased toward the second elongate arm.

Clause 7: In some examples of the medical device of clause 5 or 6, the first elongate arm is resiliently biased toward the second elongate arm by a connecting bridge between the first elongate arm and the second elongate arm.

Clause 8: In some examples of the medical device of any of clause 5, the first elongate arm is resiliently biased toward the second elongate arm by a spring member.

Clause 9: In some examples of the medical device of any of clauses 1-8, the attachment mechanism comprises an elongate arm comprising a first surface, and a second surface, wherein the elongate arm is resiliently biased to provide contact between the first surface and the second surface.

Clause 10: In some examples of the medical device of clause 9, the elongate arm comprises a first elongate arm, the attachment mechanism further comprising a second elongate arm comprising the second surface.

Clause 11: In some examples of the medical device of any of clauses 1-10, the attachment mechanism comprises at least one of an adhesive, a non-slip material, or a part of a touch fastening system.

Clause 12: In some examples of the medical device of any of clauses 1-11, the attachment mechanism comprises a portion configured to provide a snap fit with a portion of the hub when the object is between the component and the portion of the hub.

Clause 13: In some examples of the medical device of any of clauses 1-12, the attachment mechanism comprises a first portion and a second portion attached to the first portion, the first portion being configured to provide a snap fit with the second portion when the object is between the first portion and the second portion.

Clause 14: In some examples of the medical device of clause 13, the first portion is hinged to the second portion.

Clause 15: In some examples of the medical device of any of clauses 1-14, when the attachment mechanism is removably attached to the object, the attachment mechanism establishes a tension on the proximal portion when the proximal portion is subject to a pulling force in a direction away from the attachment mechanism.

Clause 16: In some examples of the medical device of any of clauses 1-15, the elongated body comprises a distal portion comprising a catheter defining a lumen, the catheter having a first maximum cross-sectional dimension, and the proximal portion comprising a push member having a second maximum cross-sectional dimension, wherein the hub is attached to a proximal end of the push member, and wherein the second maximum cross-sectional dimension is less than the first maximum cross-sectional dimension.

Clause 17: In some examples, a system comprises the medical device of any of clauses 1-16, and a catheter defining a lumen configured to receive the elongated body.

Clause 18: In some examples of the system of clause 17, the system further comprises a plurality of elongated members, wherein the lumen of the catheter is configured to simultaneously receive the plurality of elongated members, wherein the elongated body of the medical device of any of clauses 1-16 is one of the plurality of elongated members.

Clause 19: In some examples of the system of clause 17 or 18, the plurality of elongated members comprises a respective hub, wherein each hub has a respective identifying characteristic.

Clause 20: In some examples of the medical device of clauses 19, the identifying characteristic is at least one of a shape, a color, a size, a label, an alphanumeric indication, or a graphical indication.

Clause 21: In some examples, a system comprises the medical device of any of clauses 1-16 and the object, wherein the attachment mechanism is removably attached to the object.

Clause 22: In some examples, a medical device comprises an elongated body comprising a flexible proximal portion, and a hub surrounding a proximal end of the flexible proximal portion, the hub comprising an attachment mechanism configured to removably attach the hub to an object, wherein the attachment mechanism is configured to provide a nonperforating attachment to the object.

Clause 23: In some examples of the medical device of clause 22, the attachment mechanism comprises a first elongate arm and a second elongate arm movable with respect to the first elongate arm.

Clause 24: In some examples of the medical device of clauses 22 or 23, the first elongate arm is resiliently biased toward the second elongate arm.

Clause 25: In some examples of the medical device of any of clauses 22-24, the attachment mechanism comprises at least one of an adhesive, a non-slip material, or a part of a touch fastening system.

Clause 26: In some examples, a method comprises securing a hub of a medical device to an object via an attachment mechanism of the hub, the medical device comprising an elongated body comprising a proximal portion, and the hub attached to a proximal end of the proximal portion, the hub comprising the attachment mechanism configured to provide a removable attachment of the hub to the object.

Clause 27: In some examples of the method of clause 26, securing the hub of the medical device to the object comprises securing the hub of the medical device to an object within a boundary of a sterile field.

Clause 28: In some examples of the method of clause 26 or 27, the object comprises a surgical drape.

Clause 29: In some examples of the method of clauses 26-28, securing the hub of the medical device to the object comprises placing the object between a first elongate arm of the hub and a second elongate arm of the hub resiliently biased toward the first elongate arm.

Clause 30: In some examples of the method of clauses 26-29, the hub comprises an elongate arm comprising a first surface and a second surface, the elongate arm being resiliently biased to provide contact between the first surface and the second surface, and securing the hub of the medical device to the object comprises grasping the elongate arm, displacing the elongate arm relative to the second surface to provide separation between the first surface and the second surface, placing the object between the first surface and the second surface, and allowing the resilient biasing of the elongate arm to establish contact between the first surface and the object and between the second surface and the object.

Clause 31: In some examples of the method of clauses 26-30, securing the hub of the medical device to the object comprises placing the object between a first portion of the hub and a second portion of the hub configured to provide a snap fit with the first portion, and actuating the snap fit between the first portion and the second portion while the object is between the first portion and the second portion.

Clause 32: In some examples of the method of clauses 26-31, securing the hub of the medical device to the object comprises contacting a surface of the hub with the object, the surface being configured to hold the hub in place relative to the object via a traction force.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual plan view illustrating an example system including an example medical device within a sterile field boundary.

FIG. 2 is conceptual plan view illustrating an example medical device including an elongated body and an attachment mechanism.

FIG. 3A is a side view of an example medical device comprising a hub including an attachment mechanism.

FIG. 3B is a top view of the medical device of FIG. 3A.

FIG. 4 is an isometric view of the hub of FIGS. 3A and 3B.

FIG. 5A is a side view of an example of another example medical device comprising a hub including an attachment mechanism.

FIG. 5B is a top view of the medical device of FIG. 5A.

FIG. 6 is an isometric view of the hub of FIGS. 5A and 5B.

FIG. 7A is a front view of an example of another example medical device comprising a hub including an attachment mechanism.

FIG. 7B is side view of the medical device of FIG. 7A.

FIG. 8A is a front view of an example of a medical device.

FIG. 8B is side view of the medical device of FIG. 8A.

FIG. 9 is a conceptual plan view illustrating an example system including a plurality of medical devices.

FIG. 10 is a flow diagram illustrating an example method of using a medical device described herein.

### DETAILED DESCRIPTION

Example medical devices described herein include an elongated body comprising an elongated body proximal portion and a hub attached to a proximal end of the elongated body proximal portion. The hub comprises an attachment mechanism configured to provide a removable attachment of the hub to an object, such as a surgical drape. The elongated body can be, for example, a guidewire, a catheter, or a part of a catheter, such as a push member (e.g., a push wire) of a guide extension catheter. In some examples, the attachment mechanism is configured to provide a nonperforating attachment to the object. The hub may aid in wire management during medical procedures in which multiple medical devices may be present in a sterile field, e.g., multiple medical devices are proximally extending from a proximal end of a catheter. The removable attachment of the hub to an object within the sterile field may enable the elongated body proximal portion to be readily identified and easily retrieved by a clinician while simultaneously maintaining the elongated body proximal portion securely within the sterile field.

In some cases, the multiple medical devices may have similar proximal sections or other characteristics which may lead to difficulty in a clinician's ability to efficiently differentiate between each medical device, as well as delays during a procedure when medical devices within the sterile field interfere with one another. For example, wire wrap can occur as a result of elongated medical devices becoming entangled within the lumen of the catheter. This can produce environmental and ergonomic difficulties for a clinician performing the procedure.

The hub is attached to the proximal end of the elongated body. Thus, the hub is configured to attach the proximal end of the elongated body to an object. In some examples, the hub surrounds the elongated body proximal end and a section of the elongated body proximal portion. In some examples, the hub comprises a receptacle and the elongated body proximal end is received within the receptacle. The hub may be attached to elongated body proximal end using any suitable technique, such as, but not limited to, adhesives, engineering fits, fusion, friction, or welding or soldering. The connection between the hub and the elongated body may be substantially permanent, or, alternatively, may be configured to enable separation of the hub and the elongated body proximal end, such that the hub and elongated body proximal end remain substantially usable upon separation.

The hub comprises an attachment mechanism configured to provide a removable attachment to an object, such as a surgical drape residing within a sterile field. In some examples, the attachment mechanism provides a removable attachment which may be initiated and terminated manually by hand and without the use of additional tools. This may enable a clinician to relatively easily anchor a proximal end of an elongated body, such as a flexible catheter, at a location within the sterile field and substantially away from other elongated devices which may be in use during a procedure. The removable attachment may provide for effective wire management within the sterile field containing multiple elongated medical devices, as well as provide confidence the flexible elongated body is being securely maintained within the sterile field.

When multiple elongated medical devices are present in a procedure, loose trailing proximal ends can require particular attention from a clinician to ensure the various proximal ends do not become contaminated, tangled or confused with other elongated medical devices in the sterile field. Due to the flexibility and relatively long length of some medical devices, they can be difficult to securely hold in one place. For example, the proximal portions of the medical devices may inadvertently fall outside a sterile field during the medical procedure. Due to their flexibility and relatively long lengths, the clinician may require more attending clinicians simply to hold some longer instruments. The system disclosed herein provides a placement tool to keep an elongated medical device in place via an attachment mechanism at the proximal end of the medical device. The attachment mechanism is configured to help maintain the medical device within a sterile field, and the attachment at the proximal end (e.g., rather than a medial portion) may mitigate any possibility of a medical device proximal end being dragged or slowly creeping outside of a sterile field as a result of movement experienced by medial portion of the medical device. Additionally, incorporating an attachment mechanism with a hub of a medical device may provide advantage in both manufacturability and ergonomics when utilized with some medical devices, such as, but not limited to, catheters and guidewires.

The attachment mechanism of the hub is configured to attach the hub to the object using any suitable technique, such as by holding a part of the object via a mechanical grasp or via a traction force. In some examples, the attachment mechanism comprises a first elongate arm and a second elongate arm movable relative to the first elongate arm, e.g., to grasp part of an object. In some examples, one or both of the elongate arms are resiliently biased toward its counterpart by a connecting bridge of resilient material or some other component such as a spring. In some examples, the attachment mechanism may be actuated by temporarily displacing one or both elongate arms, inserting an object between the elongate arms, and enabling the resilient biasing to establish a clamping action between the first and second elongate arms and the object. For example, the first elongate arm may be resiliently biased towards the second elongate arm or the first and second elongate arms may be resiliently biased towards each other. In some examples, the first and second elongate arms are configured in a scissor type arrangement, such that a squeezing force applied to the elongate arms enables establishment of the clamping force.

In some examples, in addition to or instead of being resiliently biased, the first and second elongate arms are configured to provide a snap fit with each other when an object (e.g., the fabric of a surgical drape) is positioned between the first and second elongate arms.

In some examples, the attachment mechanism of the hub comprises a surface configured to provide a traction force (or holding force) on an object when the surface and the object are in contact. The traction force is sufficient to hold the hub substantially in place (e.g., in place or nearly in place but for some slight inconsequential movement therebetween) relative to the object. The surface may accomplish this traction force in any manner. In some examples, the surface comprises an adhesive. In some examples, the surface comprises a non-slip material such as a silicone gel. In some examples, the surface comprises a surface required by a touch fastening system, such as a hook-and-loop or dual lock fastening system. In some examples, the attachment mechanism is configured to provide a traction force that may be overcome through application of less than 20 Newtons (4.5 pound-force) to the hub, enabling the removable attachment to be relatively easily established and terminated by a clinician.

Here and elsewhere, “traction force” may refer to a force that an attachment mechanism exerts on an object when the attachment mechanism is attached to the object. “Traction force” may refer to a force which must be overcome to separate an attachment mechanism and the object when the attachment mechanism is attached to an object. In various examples, the traction force may result from placing the object between a first elongate arm and a second elongate arm of an attachment mechanism, from placing an object between a first surface and a second surface of an attachment mechanism, from placing a surface of an attachment mechanism in contact with an object, and/or from placing an object between a component of the attachment mechanism comprising a hub and a portion of the hub.

In some examples, a system may comprise the medical device and an outer catheter, such as a guide catheter, defining a lumen configured to receive the elongated body of the medical device. When the medical device is received within the lumen of the catheter, at least part of the proximal portion of the medical device and the entire hub may be positioned outside of the lumen and proximal to the proximal end of the catheter. Thus, the attachment mechanism of the hub may be used to attach the proximal end of the medical device to an object even while the medical device is positioned within the lumen of the catheter.

If the system comprises a plurality of other medical devices, such as a guidewire and one or more catheters, configured to be received within the lumen of the outer catheter at the same time as the medical device, then the attachment mechanism may help a clinician maintain separate the medical device from the plurality of other medical devices, as well as help the clinician identify the medical device from among the plurality of medical devices extending proximally from the outer catheter. If the medical device become twisted or entangled with another medical device in the catheter lumen, the medical devices can kinetically influence one another or otherwise interfere with each other. This may lead to intended movement of one medical device causing unintended movement of another medical device, or may lead to one medical device interfering with the distal advancement of another medical device through the catheter lumen. Further, proximal portions of the medical devices can become wrapped around each other, hindering adequate identification, control, or organization.

Attaching the proximal end of the medical device to an object via the attachment mechanism of the hub may enable a clinician to relatively quickly distinguish the medical device from other medical devices, as well as mitigate potential entanglement issues. For example, the clinician may know that only one or some particular types of medical devices, such as a guide extension catheter, has the hub comprising the attachment mechanism. As another example, the clinician may choose to only attach one medical device to the object via the attachment mechanism of the hub. As another example, the clinician may place the proximal end of the medical device away off to the side during a medical procedure to help keep it out of the way and help prevent entanglement between the medical device and other medical devices within the outer catheter lumen.

In some examples, a system comprising a plurality of medical device, with each medical device comprising an elongated member and a hub attached to the elongated member proximal end. The system may enable a clinician to relatively easily distinguish between multiple medical devices positioned in an outer catheter lumen and present in the sterile field. For example, the attachment mechanism may allow each of the multiple components to be attached to one or more objects at relatively disparate locations in the sterile field, increasing the efficiency of medical device identification. The disparate locations may also enable sufficient separation of any medial portions, reducing the probability of entanglements. Additionally, in some examples, at least some of the hubs of the medical devices in the plurality may have an identifying characteristic, such as, but not limited to, one or more of a shape, a color, a size, a label, an alphanumeric indication, or a graphical indication. These identifying characteristics may provide advantage to a clinician when multiple medical devices are present and must be maintained within a sterile field during a procedure. In some examples, the identifying characteristics may be unique to a particular medical device or type of medical device (e.g., a guidewire and guide extension catheter may have different identifying characteristics).

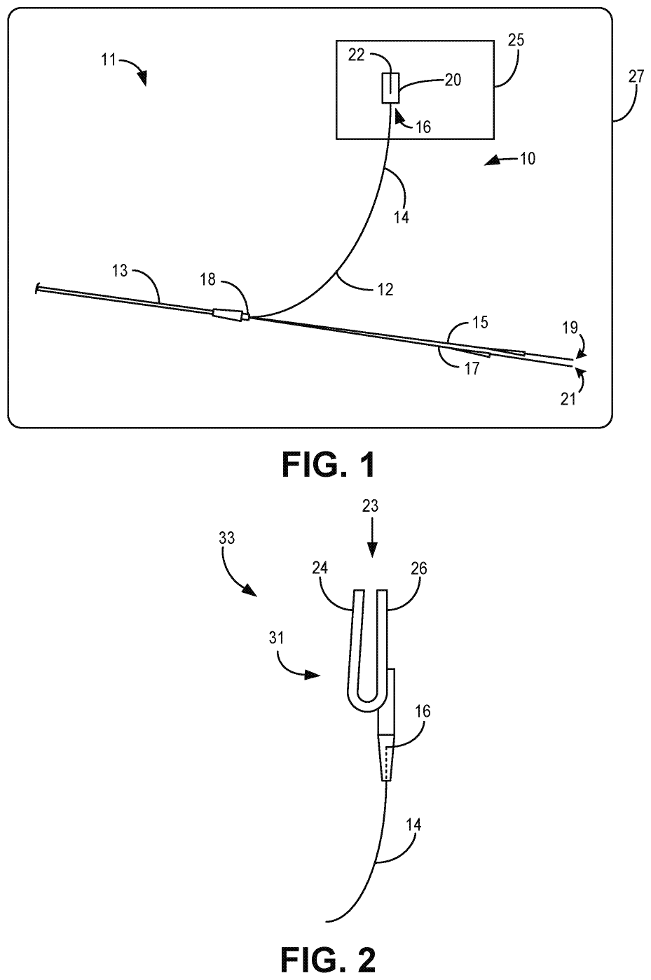

FIG. 1 illustrates an example system 11 comprising a medical device 10 comprising an elongated body 12 and a hub 20. The elongated body 12 comprises a proximal portion 14 (“elongated body proximal portion 14”) comprising a proximal end 16 (“elongated body proximal end 16”). The hub 20 is attached to the elongated body proximal end 16. The hub 20 comprises an attachment mechanism 22, shown removably attached to an object represented as a surgical drape 25. In other examples, however, the object can be another object. In many cases, the object will be located within a sterile field.

In the example shown in FIG. 1, the medical device 10 is being utilized within the system 11 comprising a catheter 13. The elongated body 12, as well as other elongated medical devices illustrated as a first medical device 15 and a second medical device 17, are simultaneously positioned within a lumen defined by the catheter 13 and proximally extend from a proximal end 18 of the catheter 13 (“catheter proximal end 18”). During some medical procedures, each of the medical devices 10, 15, 17, the surgical drape 25, and the catheter 13 are maintained within the boundary of a sterile field, depicted as a sterile field boundary 27.

As an example, an arrangement such as that depicted at FIG. 1 might be present during a percutaneous coronary intervention procedure. During some cardiac percutaneous coronary intervention procedures, a clinician may introduce a guidewire into vasculature of a patient through an access point (e.g., the femoral artery or the radial artery) to access a target treatment site within the vasculature, and introduce an outer catheter 13 (also referred to as a guide catheter in some cases) into the vasculature over the guidewire. The clinician may then introduce one or more additional catheters (e.g., a balloon catheter and/or a stent delivery catheter) into a lumen of the outer catheter to access the target site. Thus, the guidewire and the one or more additional catheters (or other medical devices) may be positioned within the outer catheter lumen at the same time, and all proximally extend from the proximal end of the outer catheter.

In some examples, the catheter 13 is an outer catheter, the medical device 10 is a guide extension catheter, the first medical device 15 may be a guidewire, and the second medical device 17 may be an interventional device, such as a balloon catheter or a stent delivery catheter. For example, the clinician may introduce a guide extension catheter into a lumen of the outer catheter 13 over the guidewire until at least a distal portion of the guide extension catheter telescopes out of the distal end of the outer catheter 13, while the proximal portion 14, which may be a push member (e.g., a push wire) in the case of a guide extension catheter, remains in the outer catheter lumen. A guide extension catheter can include, for example, a push member coupled to an elongated section via a tapered portion, with the tapered portion generally tapering from a smaller cross-sectional dimension of the push member to a larger cross-sectional dimension of the elongated section. For example, a greatest cross-sectional dimension of the push member may be smaller than a greatest cross-sectional dimension of the elongate section. The hub 20 of the medical device 10 may, therefore, be attached to a proximal end of the push member of the guide extension catheter. An example guide extension catheter is described in U.S. patent application Ser. No. 16/432,679, which was filed on Jun. 5, 2019, and is entitled, “MEDICAL CATHETER.” A guide extension catheter may be better suited for navigation through heavy tortuosity and/or calcification in a blood vessel than the outer catheter due to its flexibility and lower profile. Another medical device, such as a stent delivery catheter, may then be guided to the target site within the vasculature over the guidewire, through the outer catheter lumen, and through a lumen of the distal portion of the guide extension catheter.

These medical procedures can result in multiple medical devices proximally extending from the proximal end of the outer catheter at the same time. It can be desirable for all of the medical devices to be maintained in the sterile field established in preparation for the procedure. The multiple medical devices protruding from the outer catheter 13 may have similar proximal sections or other characteristics which may lead to difficulty in differentiating between each medical device, as well as delays during a procedure when medical devices within the sterile field interfere with one another. For example, wire wrap can occur as a result of elongated medical devices becoming entangled. For example, in the example percutaneous coronary intervention procedure discussed above, the push member of the guide extension catheter may become entangled with the guidewire, which may interfere with the ability of the stent delivery catheter or other catheter to be inserted into the guide extension catheter lumen. Wire wrap can lead to an increased likelihood of adverse issues, such as a stent catch during delivery of a stent using the outer catheter. As an example, the guidewire may wrap around the proximal push member of the guide extension catheter, such that as a stent delivery catheter is guided into the vasculature over the guidewire, the stent delivery catheter may catch on the opening to the lumen of the distal portion of the guide extension catheter.

The medical device 10 described herein is configured with a hub design that enables the hub 20, as well as the elongated body proximal end 16, to be removably attached to an object such as a surgical drape 25 in order to effectively secure and easily differentiate the elongated body 12 from amongst other extending components which may be present in the sterile field. This may provide advantages for medical device management during a procedure where a clinician is required to take steps to maintain the various components separate, in order to avoid potential entanglement. The hub design disclosed herein may provide advantage in wire management by securing elongated medical device proximal ends in place, while also enabling a clinician to easily identify and retrieve a particular elongated body.

In the example shown in FIG. 1, each of the elongated body proximal portion 14, the first medical device 15, and the second medical device 17 are maintained within a sterile field boundary 27. For illustrative purposes, FIG. 1 depicts the surgical drape 25 as an additional component within the sterile field boundary 27. However, in some cases, the surgical drape 25 may define the boundary of the sterile field 27, be one of several components of the boundary of the sterile field 27, or be one of several components maintained within a larger sterile field 27, or some other arrangement.

The attachment mechanism 22 of the hub 20 is configured to attach the hub 20 to the surgical drape 25 (or other object) using any suitable technique, such as by holding a part of the object via a mechanical grasp or via a traction force. For example, the attachment mechanism 22 may comprise a first elongate arm and a second elongate arm resiliently biased to grasp and provide a traction force on an object. As another example, in some examples, the attachment mechanism 22 may comprise a first elongate arm and a second elongate arm configured to provide a snap fit with each other when the object is between the first and second elongate arms. In some examples, in addition to or instead of being configured to hold the surgical drape 25, the attachment mechanism 22 comprises a surface configured to provide a traction force on an object when the surface and the object are in contact, allowing for relatively easy establishment and termination of the attachment.

Incorporating the attachment mechanism 22 into the hub 20 of the medical device 10 enables the attachment mechanism 22 to provide attachment to the surgical drape 25 at the proximal end of the medical device 10 (rather than a medial portion), e.g., at the elongated body proximal end 16 in the example shown in FIG. 1. This may mitigate any possibility of the proximal end being inadvertently displaced from within the boundary of the sterile field 27. Additionally, configuring the hub 20 to include the attachment mechanism 22 may provide ergonomic advantage to a clinician by allowing ready grasp of a proximal end.

FIG. 2 illustrates an example medical device 33, which is an example of the medical device of FIG. 1 and comprises the elongated body 12 and the hub 20. The hub 20 comprises an example attachment mechanism 23 and is attached to the elongated body proximal end 16. The attachment mechanism 23 is an example of the attachment mechanism 22 of FIG. 1. The attachment mechanism 23 comprises a first elongate arm 24 and a second elongate arm 26. The attachment mechanism 23 is configured to a provide removable attachment to an object, such as a surgical drape 25 or other component within the sterile field 27 (FIG. 1), between the first elongate arm 24 and the second elongate arm 26, enabling hub 20 to securely anchor the elongated body proximal portion 14 within the sterile field 27. In addition, in some examples, the attachment of the elongated body proximal portion 14 to the object may enable ready identification of the medical device 10 and relatively quick access to the medical device 10 when the elongated body proximal portion 14 is amidst other medical devices extending from the catheter 13 field.

In an example, the attachment mechanism 23 provides a removable attachment which may be terminated by hand and without the use of additional tools. For example, with the elongate arms 24, 26 providing a traction force on an object, a clinician may grasp all or some portion of the medical device 33 and detach the medical device 33 from the object using only manual manipulation of the medical device 33. For example, a clinician may terminate an attachment between the medical device 33 and the object by manually pulling the medical device 33 away from the object or pulling the object away from the medical device 33 using hand strength and a small amount of pulling force. In an example, the attachment mechanism 23 provides a removable attachment which may be established by hand and without the use of additional tools. For example, a clinician may manually actuate the elongate arms 24, 26 by placing the elongate arms 24, 26 against an object and manually urging the elongate arms 24, 26 around some portion of the object using hand strength and a small amount of pushing force, or by urging the object between the elongate arms 24, 26 using hand strength and a small amount of pushing force.

An ability to establish and terminate attachments between the medical device and an object manually and without the use of additional tools may provide ergonomic advantage to a clinician during procedures when sequential and specific manipulations of one or more instruments are required to occur in a time efficient manner. The removable attachments provided by the medical devices disclosed allows may allow the clinician to efficiently and quickly retrieve the hub attached to a particular medical device, providing for greater efficiency of sequential manual manipulations. Additionally, providing for establishment and termination of removable attachments without the use of additional tools assists in minimizing a population of items required to be tracked and maintained within an established sterile field.

In an example, the attachment mechanism 23 is configured such that when the object is positioned between the elongate arms 24, 26 and the elongate arms 24, 26 are grasping the objection, the attachment mechanism 23 establishes a tension on the elongated body 12 when the elongated body 12 is subject to a pulling force in a direction away from the attachment mechanism 23. In some examples, the removable attachment may be terminated by a human hand applying less than 20 Newtons (4.5 pound-force) to the attachment mechanism 23. In some examples, the removable attachment of the attachment mechanism 23 terminates when the elongated body 12 is subject to a pulling force less than 20 Newtons (4.5 pound-force).

As illustrated at FIG. 2, a hub 31 surrounds and is attached to the elongated body proximal end 16. The hub 31 may be attached to the elongated body proximal end 16 using an adhesive, an engineering fit, fusion, friction, welding or soldering, or some other mechanism. The attachment between the hub 31 and the elongated body proximal end 16 may be an attachment intended to be substantially permanent, and may be an attachment intended to enable separation of the hub 31 and the elongated body proximal end 16. In some examples, the attachment is such that the hub 31 and the elongated body proximal end 16 and/or the elongated body proximal portion 14 remain substantially usable upon separation.

In some examples, such as depicted at FIG. 2, the hub 31 comprises a receptacle and the elongated body proximal end 16 is inserted within the receptacle in order to provide the attachment. In some aspects, the receptacle of hub 31 and the elongated body proximal portion 14 are dimensioned such that a section of the elongated body proximal portion 14 is substantially in contact with the interior of the receptacle when hub 31 is attached to the elongated body proximal end 16. For example, the receptacle of hub 31 and a section of the elongated body proximal portion 14 may be dimensioned to provide a clearance fit, allowing movement of the elongated body proximal end 16 relative to the hub 31 when the elongated body proximal end 16 is inserted into the receptacle. The receptacle of hub 31 and the section of the elongated body proximal portion 14 may be dimensioned to provide a location or interference fit, such that mild force or high force respectively is required to move the elongated body proximal end 16 relative to the hub 20 when the elongated body proximal end 16 is inserted into the receptacle. In some aspects, the receptacle of hub 31 and the section of the elongated body proximal portion 14 may attach with a snap fit, such that some portion of the receptacle interlocks with some portion of the elongated body proximal portion 14 when the elongated body proximal end 16 is inserted into the receptacle.

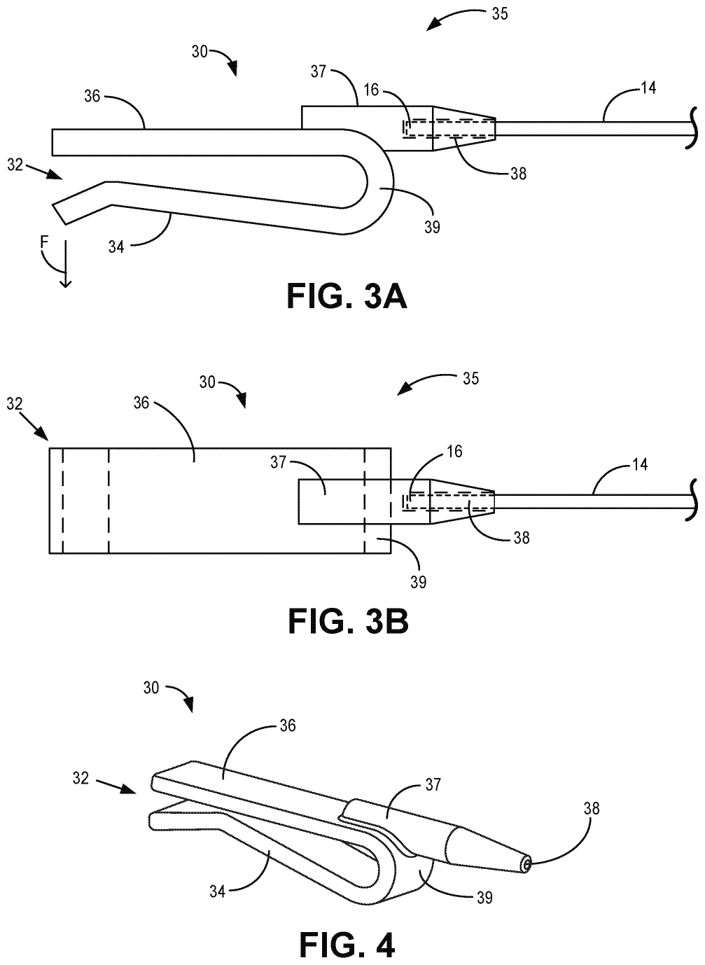

FIGS. 3A and 3B illustrate another example medical device 35 comprising a hub 30 including an attachment mechanism 32. FIG. 4 illustrates the hub 30 shown in isometric view. The medical device 35 is an example of the medical device 10 of FIG. 1, the hub 30 is an example of the hub 20 of FIG. 1, and the attachment mechanism 32 is an example of the attachment mechanism 22 of FIG. 1. FIG. 3A depicts a side view of the medical device 35 and FIG. 3B depicts a top view of the medical device 35. As illustrated at FIGS. 3A and 3B, the hub 30 comprises an attachment mechanism 32 and a receiving section 37. The hub 30 is attached to the elongated body proximal end 16 through a receiving section 37, which defines a receptacle 38. The receptacle 38 is configured to surround the elongated body proximal end 16 when the elongated body proximal end 16 is received in the receptacle 38.

In examples, the receptacle 38 may have any dimensions necessary to attach to the elongate body proximal end 16 and may attach to the elongated body proximal end 16 in any suitable manner. For example, the receptacle 38 may attach to the elongated body proximal end 16 with an adhesive, an engineering fit, fusion, friction, welding or soldering, or some other mechanism. The attachment between the receptacle 38 and the elongated body proximal end 16 may be an attachment intended to be substantially permanent, and may be an attachment intended to enable separation of the receptacle 38 and the elongated body proximal end 16. In some examples, the attachment is such that the receptacle 38 and the elongated body proximal end 16 and/or the elongated body proximal portion 14 remain substantially usable upon separation.

The attachment mechanism 32 comprises a first elongate arm 34, a second elongate arm 36, and a connecting bridge 39. The connecting bridge 39 is attached to and extends between the first elongate arm 34 and the second elongate arm 36. The first elongate arm 34 and the second elongate arm 36 are flexible elongate arms, and the first elongate arm 34 is resiliently biased toward the second elongate arm 36 by the connecting bridge 39. The first elongate arm 34 is movable with respect to the second elongate arm 36. The resilient biasing provided by the connecting bridge 39 results in a tendency of the first elongate arm 34 to return or attempt to return an initial position relative to the second elongate arm 36 when the first elongate arm 34 is temporarily displaced from the initial position by, for example, a force F acting on the first elongate arm 34 in the direction shown by the arrow in FIG. 3A. The resilient biasing further results in a traction force on an object when the first elongate arm 34 is temporarily displaced and the object is positioned between the first elongate arm 34 and the second elongate arm 36.

In an example, the traction force provided by the resilient biasing of first elongate arm 34 provides an attachment of the hub 30 to an object residing between the first elongate arm 34 and the second elongate arm 36. In some examples, the attachment may be terminated by hand without the use of additional tools. In some examples, the traction force may be overcome and the attachment terminated through application of less than 20 Newtons (4.5 pound-force) to the hub 30. In some examples, the traction force provided by the resilient biasing is such that, when a stationary object resides between the first elongate arm 34 and the second elongate arm 36, a tension generates on the elongated body 12 when the elongated body 12 is subject to a pulling force in a direction away from the object.

In some aspects, the connecting bridge 39 comprises a spring member positioned between the first elongate arm 34 and the second elongate arm 36, and the spring member provides some portion of the resilient biasing of the first elongate arm 34 toward the second elongate arm 36.

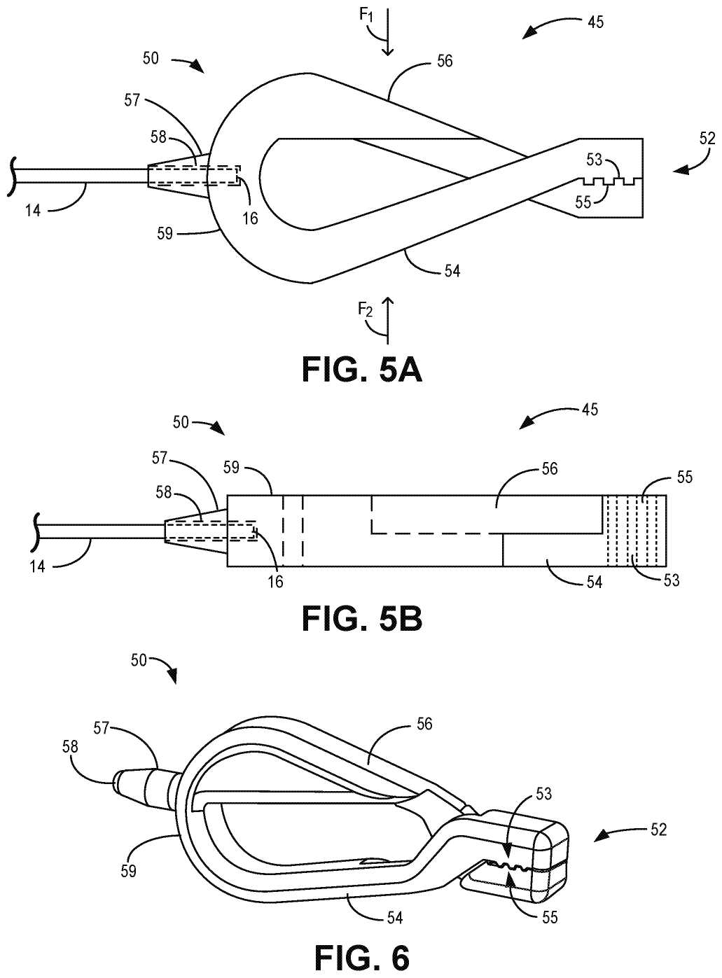

FIGS. 5A and 5B illustrate a side view and top view, respectively, of an example medical device 45 comprising a hub 50. FIG. 6 illustrates the hub 50 in isometric view. The hub 50 comprises an attachment mechanism 52 and a receiving section 57. The medical device 45 is an example of the medical device 10 of FIG. 1, the hub 50 is an example of the hub 20 of FIG. 1, and the attachment mechanism 52 is an example of the attachment mechanism 22 of FIG. 1. The hub 50 is attached to the elongated body proximal portion 14 through at least a receiving section 57 defining a receptacle 58. The receptacle 58 is configured to surround the elongated body proximal end 16 when the elongated body proximal end 16 is received in the receptacle 58. In like manner to the receptacle 38 (FIG. 3A), the receptacle 58 may have any dimensions necessary to receive and attach to the elongate body proximal end 16, may attach to the elongated body proximal end 16 in any suitable manner, and may provide attachments intended to be substantially permanent and/or enable routine separation of the receptacle 58 and the elongated body proximal end 16, e.g., without adversely affecting the structure of the receptacle 58 and the elongated body proximal end 16.

The attachment mechanism 52 comprises a first elongate arm 54 having a first surface 53. In addition, the attachment mechanism 52 comprises a second surface 55. The first elongate arm 54 is resiliently biased towards the second surface 55 by a connecting bridge 59 to provide contact between the first surface 53 and the second surface 55. The connecting bridge 59 is attached to and extends between the first elongate arm 54 and the second elongate arm 56, and resiliently biases the first elongate arm 54 and/or the second elongate arm 56 to maintain contact between the first surface 53 and the second surface 55. The first elongate arm 54 is movable with respect to the second elongate arm 56. The resilient biasing further results in a traction force on an object when the object is positioned between the first surface 53 and the second surface 55. In an example, the traction force provided by the resilient biasing of the first elongate arm 54 provides an attachment to an object residing between the first surface 53 and the second surface 55. In some examples, the attachment may be terminated by hand without the use of additional tools. In some examples, the traction force may be overcome and the attachment terminated through application of less than 20 Newtons (4.5 pound-force) to the hub 50. In some examples, the traction force provided by the resilient biasing is such that, when a fixed object resides between the first surface 53 and the second surface 55, a tension generates on the elongated body 12 when the elongated body 12 is subject to a pulling force in a direction away from the object.

In some examples, such as that depicted at FIGS. 5A and 5B, the attachment mechanism 52 comprises a second elongate arm 56 and the second elongate arm 56 comprises the second surface 55. In other examples, the second surface 55 may be defined by another portion of the hub 50. In some examples, one or more of the first elongate arm 54, the second elongate arm 56, and the connecting bridge 59 is a flexible member.

In some examples, the first elongate arm 54 and the second elongate arm 56 cross one another, and the resilient biasing provided by the connecting bridge 59 results in a tendency of the first elongate arm 54 and the second elongate arm 56 to re-establish contact between the first surface 53 and the second surface 55 when the first elongate arm 54 is temporarily displaced by, for example, a force F₁ acting in the direction shown, or by squeezing forces such as F₁ and F₂ acting in the directions shown.

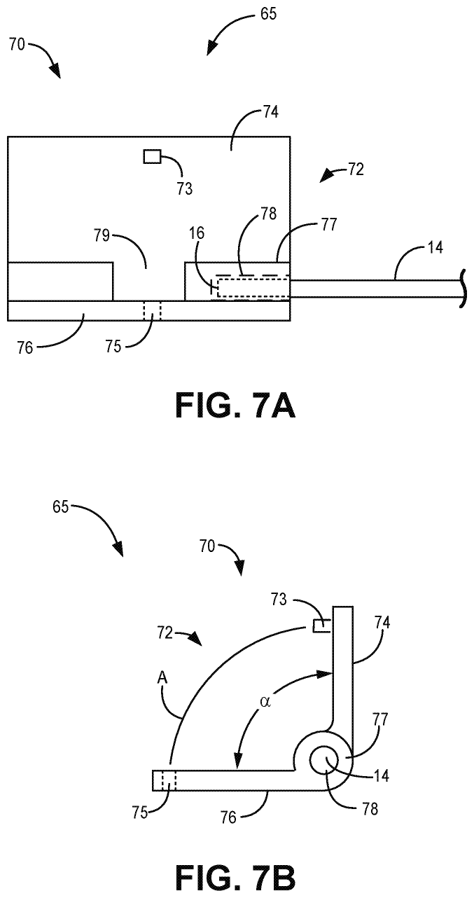

FIGS. 7A and 7B illustrate a front view and side view respectively of an example medical device 65 comprising a hub 70. The hub 70 comprises an attachment mechanism 72 and a receiving section 77. The medical device 65 is an example of the medical device 10 of FIG. 1, the hub 70 is an example of the hub 20 of FIG. 1, and the attachment mechanism 72 is an example of the attachment mechanism 22 of FIG. 1. The hub 70 is attached to an elongated body proximal end 16 through at least a receiving section 77, which defines a receptacle 78, which is similar to the receptacles 38 (FIGS. 3A, 3B, and 4), 58 (FIGS. 5A, 5B, and 6) discussed above.

The hub 70 further comprises a first portion 74 and a second portion 76, with first portion 74 configured to provide a clearance fit, location fit, or snap fit with second portion 76. The first portion 74 is movable with respect to the second portion 76. For illustration purposes, FIGS. 7A and 7B depict a subtended angle α of about 90 degrees between the first portion 74 and the second portion 76. In other examples, however, the hub 70 may be configured to provide any suitable subtended angle α between the first portion 74 and the second portion 76, such as, but not limited to, 25 degrees to 180 degrees.

In examples, the first portion 74 has a protrusion 73 and the second portion 76 has a recess 75, and the attachment mechanism 72 is configured to allow movement of the first portion 74 such that the protrusion 73 inserts into the recess 75. For example, the first portion 74 may be configured to rotate towards the second portion 76 so that the protrusion 73 substantially follows an arc A in order to insert into the recess 75. In some examples, the first portion 74 is attached to the second portion 76 by a hinge 79 enabling rotation of the first portion 74 relative to the second portion 76. In some examples, the first portion 74 and/or the second portion 76 are sufficiently flexible to allow the first portion 74 to bend along arc A.

In some examples, a protrusion 73 and a recess 75 are dimensioned to provide a clearance fit allowing the protrusion 73 and the recess 75 to move relative to each other when the protrusion 73 is inserted into the recess 75. In some examples, the protrusion 73 and the recess 75 are dimensioned to provide a location fit such that mild force (e.g., provided manually by a clinician) is required to move the protrusion 73 and the recess 75 relative to each other when the protrusion 73 is inserted into the recess 75. In some examples, the protrusion 73 and the recess 75 provide a snap fit, such that a component of the protrusion 73 interlocks with a component of the recess 75 when the protrusion 73 is inserted into the recess 75. In some examples, the protrusion 73 and the recess 75 are dimensioned to provide the clearance fit, location fit, or snap fit when some portion of an object such as a fabric is between the first portion 74 and the second portion 76. Dimensional allowance for a portion of an object allows the attachment mechanism 72 to grasp an object such as a surgical drape in a sterile field between the protrusion 73 and the recess 75.

The fit between the first portion 74 and the second portion 76 results in a traction force on an object when the object is placed between the first portion 74 and the second portion 76 and the first portion 74 is in a clearance fit, location fit, or snap fit with the second portion 76. In an example, the traction force provides an attachment to an object residing between the first portion 74 and the second portion 76. In some examples, the attachment may be terminated by hand without the use of additional tools. In some examples, the traction force may be overcome and the attachment terminated through application of less than 20 Newtons (4.5 pound-force) to the hub 70. In some examples, the traction force provided by the fit between the first portion 74 and the second portion 76 is such that, when a fixed object resides between the first portion 74 and the second portion 76, a tension generates on the elongated body 12 when the elongated body 12 is subject to a pulling force in a direction away from the object.

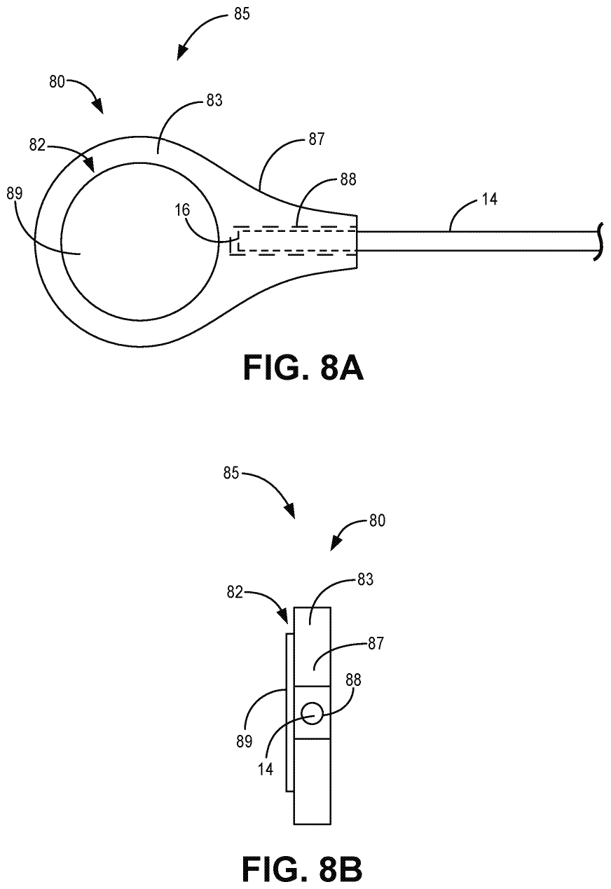

FIGS. 8A and 8B illustrate a front view and side view respectively of an example medical device 85 comprising a hub 80. The hub 80 comprises an attachment mechanism 82 and a receiving section 87. The medical device 85 is an example of the medical device 10 of FIG. 1, the hub 80 is an example of the hub 20 of FIG. 1, and the attachment mechanism 82 is an example of the attachment mechanism 22 of FIG. 1. The hub 80 is attached to the elongated body proximal end 16 through at least a receiving section 87, which comprises a receptacle 88, which is similar to the receptacles 38 (FIGS. 3A, 3B, and 4), 58 (FIGS. 5A, 5B, and 6) discussed above.

The attachment mechanism 82 comprises a surface 89 configured to provide a traction force on an object when the surface 89 and the object are in contact. The surface 89 may accomplish this traction force in any manner. In some examples, the surface 89 comprises an adhesive and the adhesive provides the traction force when contacting the object. In some examples, the surface 89 comprises a non-slip material such as a silicone gel. In some examples, the hub 80 comprises a hub body 83, and the surface 89 is attached to the hub body 83. In some examples, the hub body 83 has a first coefficient of friction with an object when in planer contact with the object, and the surface 89 comprises a material or coating having a second coefficient of friction when in planer contact with the object, and the second coefficient of friction exceeds the first coefficient of friction. In some examples, the surface 89 comprises a part of a touch fastening system. For example, in a hook-and-loop fastening system (such as VELCRO® brand fasteners, made available by Velcro Company of Middlewich, United Kingdom), the surface 89 may comprise hooks, loops, or a combination of hooks and loops. In a dual lock fastening system in which a first feature (such as a mushroom shaped stem) on a first surface locks with a second feature (such as a recess) on a second feature, the surface 89 may comprise the first feature, the second feature, or a combination of the first feature and the second feature.

The traction force provided by the surface 89 on an object provides an attachment of the hub 80 to the object. Thus, the traction force provided by the surface 89 may depend on the target object to which the hub 80 is intended to be attached. In some examples, the attachment may be terminated by hand without the use of additional tools. In some examples, the traction force may be overcome and the attachment terminated through application of less than 20 Newtons (4.5 pound-force) to the hub 80. In some examples, the traction force on the object provided by the surface 89 is such that a tension generates on the elongated body 12 when the elongated body 12 is subject to a pulling force in a direction away from the object.

In some examples, such that depicted in FIG. 1, the system 11 comprises the elongated body 12 comprising the flexible proximal portion 14 and the elongated body proximal end 16. The elongated body 12 further comprises a medial portion (not shown) displaced from the elongated body proximal end 16 in a distal direction away from the elongated body proximal end 16. The hub 20 is attached to the elongated body proximal end 16, and the hub 20 is configured to provide a removable attachment to an object, such as the surgical drape 25 residing within the sterile field boundary 27. The system 11 further comprises the catheter 13 having a lumen, and the medial portion of the elongated body 12 within the lumen. Such a system enables the removable attachment of the hub 20 to an object to help anchor the hub 20 and the elongated body 12 within a sterile field, such as the sterile field established within the boundary 27. The nature of the removal attachment as described herein may enable a clinician to easily retrieve the hub 20 and the elongated body 12 by hand and with confidence that the hub 20 and the elongated body 12 have remained within the sterile field.

In some examples, the system 11 comprises a plurality of medical devices, with the medical device 10 as one of a plurality. Each medical device of the plurality comprises a medial portion displaced from the respective proximal end in a distal direction away from the proximal end. Each medical device may be configured to be received within the lumen of the catheter 13 simultaneously, such that a medial portion of each medical device is within the lumen and the proximal end of each medical device is outside the lumen of the catheter. For example, at FIG. 1, the plurality of medical devices comprises the first medical device 15 having the proximal end 19, the second medical device 17 having the proximal end 21, and the medical device 10 including the elongated body 12 having the elongated body proximal end 16 attached to the hub 20. As depicted in FIG. 1, medial portions of the first medical device 15, the second medical device 17, and the medical device 10 (e.g., a medial portion of the elongated body 12) are within the lumen of the catheter 13, while the proximal end 19 of the first medical device 15, the proximal end 21 of the second medical device 17, and the elongated body proximal end 16 are outside the lumen.

In some examples, the hub 20 of the medical device 10 and, in some examples, hubs of the first medical device 15 and/or the second medical device 17, comprise a visible identifying characteristic that enables a clinician to relatively quickly and easily identify the respective medical device amongst multiple components with may be present in the sterile field. This may aid in medical device management when multiple medical devices are being used in a medical procedure. The removable attachment provided by the attachment mechanism 22 of the hub 20 may enable the elongated body proximal portion 14 to be readily identified and easily retrieved by a clinician while simultaneously maintaining the elongated body proximal portion 14 securely within the sterile field 27.

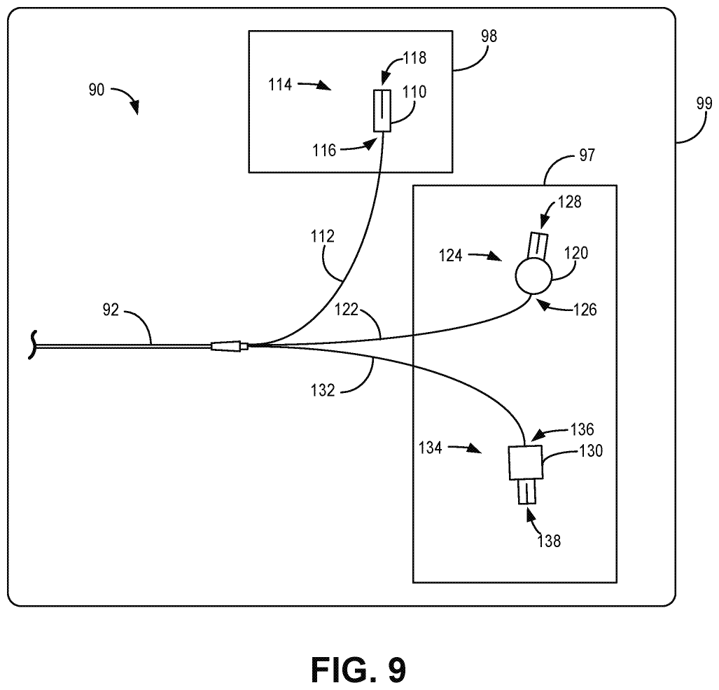

In some examples, a system comprises a plurality of medical devices, with each medical device comprising an elongated body comprising a hub attached to the proximal end of the elongated body, where each hub is configured to be removably attached to an object. For example, FIG. 9 illustrates an example system 90 comprising a first medical device 114, a second medical device 124, and a third medical device 134. The first medical device 114 comprises a first elongated body 112 and a first hub 110, with the first hub 110 attached to the proximal end 116 of the first elongated body 112. The first hub 110 further comprises a first attachment mechanism 118 configured to be removably attached to an object. The second medical device 124 comprises a second elongated body 122 and a second hub 120, with the second hub 120 attached to the proximal end 126 of the second elongated body 122. The second hub 120 further comprises a second attachment mechanism 128 configured to be removably attached to an object. A third medical device 134 comprises a third elongated body 132 and a third hub 130, with the third hub 130 attached to a proximal end 136 of the third elongated body 132. The third hub 130 further comprises a third attachment mechanism 138 configured to be removably attached to an object.

FIG. 9 further illustrates a sterile field boundary 99. The first medical device 114, the second medical device 124, and the third medical device 134 are within the sterile field boundary 99, as is a first surgical drape 98 and a second surgical drape 97. At FIG. 9, the first medical device 114 is removably attached to the first surgical drape 98 by the first attachment mechanism 118. The second medical device 124 and the third medical device 134 are removably attached to the second surgical drape 97 by the second attachment mechanism 128 and the third attachment mechanism 138 respectively. Additionally, each of the first medical device 114, the second medical device 124, and the third medical device 134 have a respective identifying characteristic, which may be unique to the respective medical device in some examples or unique to the type of medical device (e.g., guidewires may have the same identifying characteristic). FIG. 9 depicts the characteristic as a shape of each hub within the plurality. In other examples, in addition to the shape of the hub, a color, a size, an alphanumeric indication, a graphical indication, or any other visible characteristic may be utilized to identify a medical device within a plurality of medical devices. These identifying characteristics may provide advantage to a clinician when multiple medical devices are used during a medical procedure. In contrast, in examples in which the multiple medical devices used during a medical procedure and extending from a common catheter have similar proximal sections, a clinician may have difficulty differentiating between the medical devices, which may lead to a longer duration medical procedure.

Use of the medical system disclosed with each of multiple medical devices present may alleviate potential wire and medical device management issues, by providing a hub for each medical device that enables attachment at relatively disparate locations in the sterile field. This may increase the efficiency of correct retrieval and identification as well as reduce the probability of entanglements.

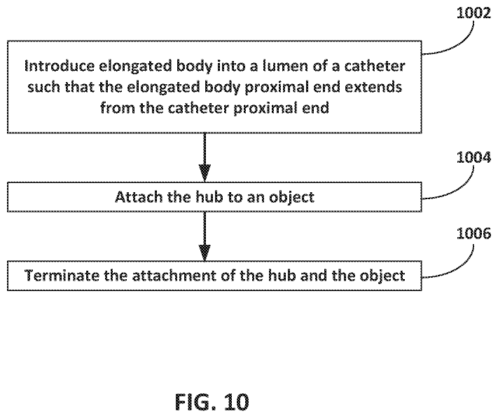

FIG. 10 is a flow diagram of an example method of using system 11 (FIG. 1). In some examples, the elongated body proximal end 16 may be pre-attached to the hub 20 by a distributor of the medical device 10. In other examples, a clinician may attach the hub 20 to the elongated body proximal end 16. The steps of the method are presented in a specific order for explanatory purposes; the steps may be conducted in any suitable order in other examples.

The clinician may introduce the elongated body 12 into a lumen of the catheter 13 such that the elongated body proximal end 16 extends proximally from the catheter proximal end 18 (1002). The clinician may advance the elongated body 12 toward a distal end of the catheter 13 by providing a pushing force to the elongated body 12 in a distal direction. The clinician may subject the elongated body to motion in the distal or proximal direction when the elongated body 12 is within the catheter 13. In some medical procedures, the clinician may introduce the elongated body 12 and the hub 20 within the boundary 27 of the sterile field.

In some examples, the clinician may also introduce additional elongated members into the lumen of the catheter 13, such that a plurality of elongated members extends from the catheter proximal end 18. The additional elongated members may be pre-attached to a respective hub, or the clinician may attach a hub to one or more of the additional elongated members.

To help with device management, the clinician may attach the hub 20 of the elongated body 12 to an object 25 using the attachment mechanism 22 (1004). For example, the clinician may attach the hub 20 to a sterile object 25 within the boundary 27 of the sterile field using the attachment mechanism 22, while maintaining the elongated body 12 and the hub 20 within the boundary of the sterile field. In some examples, the clinician may attach the hub 20 to the sterile object 25 manually. If other elongated members used with the catheter 13 also include hubs defining respective attachment mechanisms, the clinician may also attach one or more of the hubs attached to the additional elongated members to the sterile object 25 or another sterile object in the sterile field.

During or after a medical procedure, the clinician may terminate the attachment between the hub 20 and the object 25 (1006), e.g., to enable the elongated body 12 to be proximally withdrawn from the lumen of the catheter 13. For example, the clinician may terminate the attachment manually. The clinician may also terminate the attachment between any of the one or more hubs attached to any additional elongated bodies.

Various examples have been described. These and other examples are within the scope of the following claims.

What is claimed is:

1. A medical device comprising:
an elongated body comprising a proximal portion; and
a hub attached to a proximal end of the proximal portion, the hub comprising an attachment mechanism configured to provide a removable attachment of the hub to an object.

2. The medical device of claim 1, wherein the attachment mechanism is configured to provide a nonperforating attachment to the object.

3. The medical device of claim 1, wherein the elongated body comprises a guide extension catheter and the proximal portion comprises a push member.

4. The medical device of claim 1, wherein the hub surrounds the proximal end of the proximal portion.

5. The medical device of claim 1, wherein the attachment mechanism comprises a first elongate arm and a second elongate arm movable relative to the first elongate arm.

6. The medical device of claim 5, wherein the first elongate arm is resiliently biased toward the second elongate arm.

7. The medical device of claim 1, wherein the attachment mechanism comprises:
an elongate arm comprising a first surface; and
a second surface, wherein the elongate arm is resiliently biased to provide contact between the first surface and the second surface.

8. The medical device of claim 1, wherein the attachment mechanism comprises at least one of an adhesive, a non-slip material, or a part of a touch fastening system.

9. The medical device of claim 1, wherein the attachment mechanism comprises a portion configured to provide a snap fit with a portion of the hub when the object is between the component and the portion of the hub.

10. The medical device of claim 1, wherein the attachment mechanism comprises a first portion and a second portion attached to the first portion, the first portion being configured to provide a snap fit with the second portion when the object is between the first portion and the second portion.

11. The medical device of claim 1, wherein when the attachment mechanism is removably attached to the object, the attachment mechanism establishes a tension on the proximal portion when the proximal portion is subject to a pulling force in a direction away from the attachment mechanism.

12. The medical device of claim 1, wherein the elongated body comprises:
a distal portion comprising a catheter defining a lumen, the catheter having a first maximum cross-sectional dimension; and
the proximal portion comprising a push member having a second maximum cross-sectional dimension,
wherein the hub is attached to a proximal end of the push member, and
wherein the second maximum cross-sectional dimension is less than the first maximum cross-sectional dimension.

13. A system comprising:
the medical device of claim 1; and
a catheter defining a lumen configured to receive the elongated body.

14. The system of claim 13, further comprising a plurality of elongated members, wherein the lumen of the catheter is configured to simultaneously receive the plurality of elongated members, wherein the elongated body of the medical device of claim 1 is one of the plurality of elongated members.

15. The system of claim 14, wherein the plurality of elongated members comprises a respective hub, wherein each hub has a respective identifying characteristic.

16. The system of claim 15, wherein the identifying characteristic is at least one of a shape, a color, a size, a label, an alphanumeric indication, or a graphical indication.

17. A system comprising:
the medical device of claim 1; and
the object, wherein the attachment mechanism is removably attached to the object.

18. A medical device comprising:
an elongated body comprising a flexible proximal portion; and
a hub surrounding a proximal end of the flexible proximal portion, the hub comprising an attachment mechanism configured to removably attach the hub to an object, wherein the attachment mechanism is configured to provide a nonperforating attachment to the object.

19. The medical device of claim 18, wherein the attachment mechanism comprises a first elongate arm and a second elongate arm movable with respect to the first elongate arm.

20. The medical device of claim 18, wherein the attachment mechanism comprises at least one of an adhesive, a non-slip material, or a part of a touch fastening system.

21. A method comprising:
securing a hub of a medical device to an object via an attachment mechanism of the hub, the medical device comprising:
an elongated body comprising a proximal portion; and
the hub attached to a proximal end of the proximal portion, the hub comprising the attachment mechanism configured to provide a removable attachment of the hub to the object.

22. The method of claim 21, wherein securing the hub of the medical device to the object comprises securing the hub of the medical device to an object within a boundary of a sterile field.

23. The method of claim 21, wherein securing the hub of the medical device to the object comprises placing the object between a first elongate arm of the hub and a second elongate arm of the hub resiliently biased toward the first elongate arm.

24. The method of claim 21, wherein securing the hub of the medical device to the object comprises:
placing the object between a first portion of the hub and a second portion of the hub configured to provide a snap fit with the first portion; and
actuating the snap fit between the first portion and the second portion while the object is between the first portion and the second portion.

25. The method of claim 21, wherein securing the hub of the medical device to the object comprises contacting a surface of the hub with the object, the surface being configured to hold the hub in place relative to the object via a traction force.
